SEQUENCE LISTING (32 sequences)

SEQ ID NO: 1 - DNA, 18 bp, Artificial sequence
  oligonucleotide PCR primer
acgccggagaatacaatg

SEQ ID NO: 2 - DNA, 18 bp, Artificial sequence
  oligonucleotide PCR primer
caacggcacaatctcatg

SEQ ID NO: 3 - DNA, 16 bp, Artificial sequence
  oligonucleotide PCR primer
gacacccagttcaaaa

SEQ ID NO: 4 - DNA, 18 bp, Artificial sequence
  oligonucleotide  PCR primer
ctcaacttcttcccgaca

SEQ ID NO: 5 - DNA, 18 bp, Artificial sequence
  oligonucleotide PCR primer
ggaacaagcggacctcat

SEQ ID NO: 6 - DNA, 18 bp, Artificial sequence
  oligonucleotide PCR primer
tctcagcgatcttgcctt

SEQ ID NO: 7 - DNA, 18 bp, Artificial sequence
  oligonucleotide PCR primer
gctggcaggcgtaacact

SEQ ID NO: 8 - DNA, 21 bp, Artificial sequence
  oligonucleotide PCR primer
cggtttgaggagttgtagaag

SEQ ID NO: 9 - DNA, 18 bp, Artificial sequence
  oligonucleotide PCR primer
cgtcttgaaccacacagc

SEQ ID NO: 10 - DNA, 18 bp, Artificial sequence
  oligonucleotide PCR primer
gcaaagtctccctcctct

SEQ ID NO: 11 - DNA, 18 bp, Artificial sequence
  oligonucleotide PCR primer
ccagggtagaggaaacaa

SEQ ID NO: 12 - DNA, 18 bp, Artificial sequence
  oligonucleotide PCR primer
tcgaagaagaccgctggt

SEQ ID NO: 13 - DNA, 19 bp, Artificial sequence
  oligonucleotide PCR primer
aagatgaaggaaatgagtg

SEQ ID NO: 14 - DNA, 18 bp, Artificial sequence
  oligonucleotide PCR primer
catcttttctggtctcgg

SEQ ID NO: 15 - DNA, 18 bp, Artificial sequence
  oligonucleotide PCR primer
ggaccaagggcaagtaag

SEQ ID NO: 16 - DNA, 18 bp, Artificial sequence
  oligonucleotide PCR primer
agcccatcctcctctgtg

SEQ ID NO: 17 - DNA, 20 bp, Artificial sequence
  oligonucleotide PCR primer
aatgacaatatctgagactc

SEQ ID NO: 18 - DNA, 18 bp, Artificial sequence
  oligonucleotide PCR primer
accacattctttagcctc

SEQ ID NO: 19 - DNA, 19 bp, Artificial sequence
  oligonucleotide PCR primer
ctttgactctgttctcggt

SEQ ID NO: 20 - DNA, 18 bp, Artificial sequence
  oligonucleotide
gttgtcagtttctccgcc

SEQ ID NO: 21 - DNA, 20 bp, Artificial sequence
  oligonucleotide PCR primer
tcaagcggataagagtcact

SEQ ID NO: 22 - DNA, 18 bp, Artificial sequence
  oligonucleotide PCR primer
ctcgtccgggttaatgct

SEQ ID NO: 23 - DNA, 18 bp, Artificial sequence
  oligonucleotide PCR primer
ggagcgatttgtctggtt

SEQ ID NO: 24 - DNA, 18 bp, Artificial sequence
  oligonucleotide PCR primer
tgatgactcgcgcttact

SEQ ID NO: 25 - DNA, 18 bp, Artificial sequence
  oligonucleotide PCR primer
ggagcgatttgtctggtt

SEQ ID NO: 26 - DNA, 18 bp, Artificial sequence
  oligonucleotide PCR primer
tgatgactcgcgcttact

SEQ ID NO: 27 - DNA, 264 bp, Artificial sequence
  transit peptide encoding sequence
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccggtggtcgcgtcaactgcatgcaggcc

SEQ ID NO: 28 - protein, 88 aa, Artificial sequence
  transit peptide encoding sequence
MASSMLSSATMVASPAQATMVAPFNGLKSSAAFPATRKANNDITSITSNGGRVNCMQVWPPIGKKKFETLSYLPDLTDSGGRVNCMQA

SEQ ID NO: 29 - DNA, 174 bp, Artificial sequence
  transit peptide sequence
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggcc

SEQ ID NO: 30 - protein, 58 aa, Artificial sequence
  transit peptide sequence
MASSMLSSATMVASPAQATMVAPFNGLKSSAAFPATRKANNDITSITSNGGRVNCMQA

SEQ ID NO: 31 - DNA, 294 bp, Arabidopsis thaliana
tcatttctcatcataacaaagagagagaaaaaaactatggaattgacactgaattcctcgagttctcttatcaaacgtaaagatgccaagagttctagaaaccaagaaagttcctccaacaacatgacctttgcgaagatgaagccgccaacatatcagttccaagcaaagaactcggttaaggaaatgaagttcactcacgagaagaccttcacgccagaaggtgaaacccttgagaaatgggagaagctccacgttctctcatacccacactccaagaacgacgctagcgtt

SEQ ID NO: 32 - protein, 86 aa, Arabidopsis thaliana
MELTLNSSSSLIKRKDAKSSRNQESSSNNMTFAKMKPPTYQFQAKNSVKEMKFTHEKTFTPEGETLEKWEKLHVLSYPHSKNDASV

(19) United States
(12) Patent Application Publication
(10) Pub. No.: US 2004/0091900 A1
(43) Pub. Date: May 13, 2004

(54) Materials and methods for providing plants with increased resistance to environmental stress

(76) Inventors: Charles L. Guy, Gainesville, FL (US); Fatma Kaplan, Gainesville, FL (US); Dong Yul Sung, San Diego, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/602,395

(22) Filed: Jun. 23, 2003

Related U.S. Application Data
(60) Provisional application No. 60/390,384, filed on Jun. 21, 2002.

(51) Int. Cl. C12Q 1/68; A01H 1/00; C12N 15/82

(52) U.S. Cl. 435/6; 800/284

(57) ABSTRACT

The subject invention pertains to materials and methods for protecting plants and plant organelles, such as chloroplasts, during thermal (heat and cold) stress, and other forms of environmental stress such as water and salt stress. In one embodiment, a plant is transformed with a polynucleotide that encodes a protein that produces, catalyzes the synthesis of or results in the production of maltose or a maltose alcohol. In an exemplified embodiment, the polynucleotide encodes a β-amylase enzyme that is localized at the chloroplast. The subject invention also concerns plants and plant tissue transformed with a polynucleotide that encodes a protein that produces or results in the production of maltose or a maltose alcohol.

### CROSS-REFERENCE TO A RELATED APPLICATION

This application claims the benefit of U.S. Provisional Application Serial No. 60/390,384, filed Jun. 21, 2002.

### BACKGROUND OF INVENTION

A variety of factors are involved in starch degradation in plants. Diastase is a group of enzymes that catalyze the breakdown of carbohydrates. Diastase enzymes include alpha-amylase, beta-amylase, starch phosphorylase, and starch debranching enzyme (DBE) or R-enzyme. Maltose is either a major product or initial product of starch degradation by such enzymes.

Alpha-amylase (α-amylase) hydrolyzes starch, glycogen, and related polysaccharides by randomly cleaving internal α-1,4 glucosidic linkages. It is widely distributed in plants and has a major role in the utilization of polysaccharides. Under anoxic conditions, rice seeds are able to induce α-amylase.

Beta-amylase (β-amylase) is an exoamylase that hydrolyses α-1,4 glycosidic linkages of polyglucan chains at the non reducing end to produce maltose (4-O-α-D-Glucopyranosyl-β-D-glucose) (see Scheme 1 below). The primary physiological role of β-amylase is considered to be in starch breakdown (Beck and Ziegler 1989).

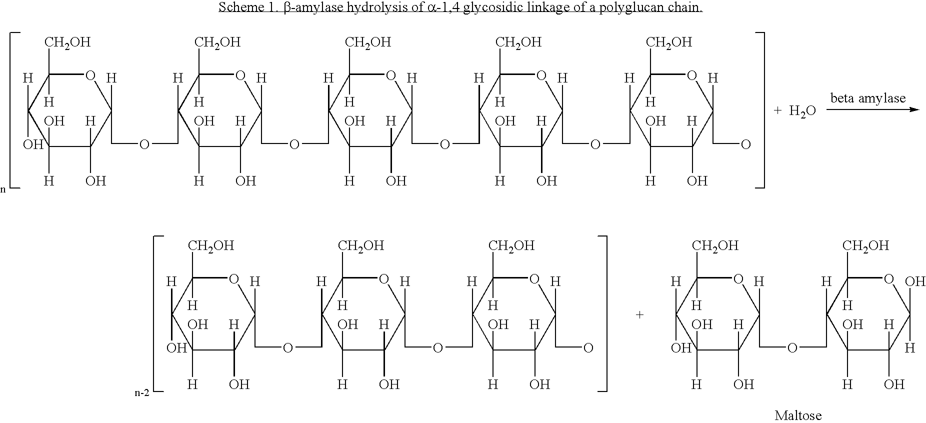

The complete physiological role of β-amylase in starch breakdown remains unclear because this enzyme is considered by some, based on in vitro studies to be unable to attack natives starch without prior digestion by other amylolytic enzymes like α-amylase (Beck and Ziegler, 1989). Current models of β-amylase function favor its action to be predominantly on polyglucan chains. β-amylase randomly encounters polyglucan chains and. hydrolyzes sequentially after complexing with the polymeric glucan chains (Adachi et al., 1998).

The enzyme β-amylase and its activity is found in seeds, tubers, and leaves; everywhere starch is present in plants (Avigad and Dey 1997). In leaves, this enzyme has been localized to phloem sieve tubes (Wang et al., 1995), the cytoplasm of mesophyll cells (Monroe and Preiss, 1990), and in the stroma of chloroplasts (Scheidig et al., 2002; Lao et al., 1999). One Arabidopsis β-amylase, designated, ct-Bmy (AJ250341; BMY8), has been biochemically localized to the chloroplast stroma (Lao et al. 1999) in import studies with isolated pea chloroplasts, and confirmed by accumulation of a β-amylase-GFP fusion protein in Arabidopsis chloroplasts (Lao et al. 1999). Database searches have revealed that Arabidopsis contains nine β-amylases, designated BMY1 to BMY9. BMY1, which is also called ram1, accounts for more than 80% of total β-amylase activity in Arabidopsis and is localized to the vacuole or the secretory pathway in mesophyll cells. BMY2 to BMY6 are localized to the cytosol, based on sequence analysis with Target P. BMY7, BMY8, and BMY9 contain a putative chloroplast targeting peptide.

Beta-amylase expression is induced under stress conditions such as drought (Todaka et al., 2000), cold (Nielsen et al., 1997), and heat stress (Dreier et al., 1995). When Arabidopsis plants are cold stressed at 4° C. for 12 hours, β-amylase (AJ25034; ct-Bmy or BMY8) expression increases about 14-fold (Sung 2001) and induction can occur as early as 2 hr after exposure to cold stress (Seki et al. 2001). During water stress, β-amylase activity increases and is followed by an increase in the free sugars, sucrose and maltose in cucumber cotyledons (Todaka et al., 2000). Similarly, when pearl millet, maize (Datta et al., 1999) and barley (Dreier et al., 1995) are exposed to water and salt stress, β-amylase activity is similarly induced. The increase in the β-amylase activity is correlated with increased β-amylase protein. When potato tuber storage temperature is reduced from 20° C. to 5° C. or 3° C., β-amylase activity is increased four to five fold within 10 days, whereas the activity of α-glycosidase, which hydrolyzes α1,1; α1,2; α1,3; α1,4, and α1,6 glycosidic bonds, and endoamylase, which hydrolyzes internal α1,4 glycosidic linkages of starch, is not changed. However, the role of β-amylase under temperature shock is unknown. Chloroplast localized β-amylase has been shown to be strongly stress induced in a number of cold and heat stress related microarray studies (Seki et al., 2001; Sung 2001; Seki et al., 2002; Fowler and Thomashow 2002; Kreps et al., 2002).

Additional enzymes that produce maltose during the breakdown of starch include starch phosphorylase and starch DBE. Starch phosphorylase cleaves and phosphorylates terminal glucose residues in starch, only cleaving α-1,4 linked residues and not α-1,6 or α-1,4 close to α-1,6 linkages. Starch DBE hydrolyzes α-1,6 linkages in glucans. In particular, DBE is active on amylopectin, α-limit dextrins, and phosphorylase-limit dextrins.

Compatible solutes (osmoprotectants, osmolytes) are, low molecular weight organic molecules that accumulate under stress conditions. Examples of compatible solutes include trehalose, sucrose, glucose, and fructose. They help to stabilize proteins and membranes and contribute to cell osmotic pressure under stress conditions (Yancey et al., 1982). There are three general types of osmoprotectants: methylamines (betaines), amino acids (proline), and polyols (glycerol, sucrose) (Yancey et al., 1982).

In many plants, sucrose is a compatible solute that is considered to stabilize membranes and proteins during temperature stress and water stress (Santarius, 1973). Sucrose accumulates in a wide range of plants during temperature stress (Wanner and Juntilla, 1999; Krapp and Stitt, 1995; Strand et al., 1999; Strand et al., 1997; Guy, et al., 1992). The activity and transcripts of sucrose biosynthetic enzyme, sucrose phosphate synthase, (SPS), are known to increase under temperature stress (Guy et al., 1992; Strand et al., 1997). When Arabidopsis plants are exposed to low temperature (5° C.), within a few hours sugar accumulation follows consisting primarily of sucrose, glucose, and fructose (Guy et al., 1992; Wanner and Juntilla, 1999), phosphorylated hexose intermediates (Strand et al., 1997; Strand et al., 1999; Krapp and Stitt, 1995). The increase in sucrose is well correlated with an increased expression (Strand et al., 1997) and activity of SPS (Guy et al., 1992).

Proline is another compatible solute that accumulates under stress conditions such as salt, dehydration, and cold stress (Verbruggen et al., 1993; Yoshiba et al 1995; Xin and Browse, 1998; Wanner and Juntilla, 1999; Gilmour et al., 2000). Δ¹ pyrroline-5-carboxylate synthetase (P5CS) catalyzes the first two steps in proline biosynthesis in plants (Hu et. al., 1992). It is suggested that P5CS plays a key role in proline biosynthesis under osmotic stress, and catalyzes the major regulated step (Yoshiba, et al., 1995; Strizhov et al., 1997).

Acute temperature shock is likely to induce deleterious effects in all parts of a cell. Since compatible solutes contribute to acquired thermotolerance (Singer and Lindquist, 1998), as well as acquired freezing tolerance mechanisms (Guy, 1990), then accumulation of one or more compatible solutes in the chloroplast plays a critical, part of a multi-faceted network of molecules, stress proteins, biochemical transformations and physiological adjustments that collaborate to preserve the viability of an organelle, a cell, a tissue or an organism during transient periods of stress that would be otherwise lethal (Thomashow, 1999).

### BRIEF SUMMARY OF THE INVENTION

The subject invention pertains to materials and methods for providing plants, plant tissue, plant cells, and plant organelles, such as chloroplasts, with increased resistance to thermal (heat and/or cold) stress, and other forms of environmental stress such as water, salt stress, nutritional stress, aerobic and anaerobic stress, and wounding. In one embodiment, a plant is transformed with a polynucleotide that encodes a protein that produces, or catalyzes the synthesis of, or results in the production of maltose or a maltose alcohol. In an exemplified embodiment, the polynucleotide encodes a diastase enzyme, or an enzymatically active fragment thereof.

The subject invention also concerns plants, plant tissue and plant cells transformed with or bred to contain a polynucleotide that encodes, a protein that produces, or catalyzes the synthesis of, or results in the production of maltose or a maltose alcohol. In an exemplified embodiment, the polynucleotide encodes a β-amylase enzyme. In one embodiment, the polynucleotide is overexpressed in the plant in order to elevate the level of maltose or maltose alcohol in a plant.

### BRIEF DESCRIPTION OF THE SEQUENCES

SEQ ID NO. 1 is an oligonucleotide used in PCR amplification.

SEQ ID NO. 2 is an oligonucleotide used in PCR amplification.

SEQ ID NO. 3 is an oligonucleotide used in PCR amplification

SEQ ID NO. 4 is an oligonucleotide used in PCR amplification.

SEQ ID NO. 5 is an oligonucleotide used in PCR amplification.

SEQ ID NO. 6 is an oligonucleotide used in PCR amplification.

SEQ ID NO. 7 is an oligonucleotide used in PCR amplification.

SEQ ID NO. 8 is an oligonucleotide used in PCR amplification.

SEQ ID NO. 9 is an oligonucleotide used in PCR amplification.

SEQ ID NO. 10 is an oligonucleotide used in PCR amplification.

SEQ ID NO. 11 is an oligonucleotide used in PCR amplification.

SEQ ID NO. 12 is an oligonucleotide used in PCR amplification.

SEQ ID NO. 13 is an oligonucleotide used in PCR amplification.

SEQ ID NO. 14 is an oligonucleotide used in PCR amplification.

SEQ ID NO. 15 is an oligonucleotide used in PCR amplification.

SEQ ID NO. 16 is an oligonucleotide used in PCR amplification.

SEQ ID NO. 17 is an oligonucleotide used in PCR amplification.

SEQ ID NO. 19 is an oligonucleotide used in PCR amplification.

SEQ ID NO. 20 is an oligonucleotide used in PCR amplification.

SEQ ID NO. 21 is an oligonucleotide used in PCR amplification.

SEQ ID NO. 21 is an oligonucleotide used in PCR amplification.

SEQ ID NO. 22 is an oligonucleotide used in PCR amplification.

SEQ ID NO. 23 is an oligonucleotide used in PCR amplification.

SEQ ID NO. 24 is an oligonucleotide used in PCR amplification.

SEQ ID NO. 25 is an oligonucleotide used in PCR amplification.

SEQ ID NO. 26 is an oligonucleotide used in PCR amplification.

SEQ ID NO. 27 is a transit peptide encoding sequence that encodes the amino acid sequence shown in SEQ ID NO. 28 that can be used according to the present invention.

SEQ ID NO. 28 is a transit peptide sequence that can be used according to the present invention.

SEQ ID NO. 29 is a transit peptide encoding sequence that encodes the amino acid sequence shown in. SEQ ID NO. 30 that can be used according to the present invention.

SEQ ID NO. 30 is a transit peptide sequence that can be used according to the present invention.

SEQ ID NO. 31 is a transit peptide encoding sequence that encodes the amino acid sequence shown in SEQ ID NO. 32 that can be used according to the present invention.

SEQ ID NO. 32 is a transit peptide sequence that can be used according to the present invention.

### BRIEF DESCRIPTION OF DRAWINGS

FIG. 1 shows RT-PCR analysis of selected genes following heat and cold shock. Arrow head shows 18S rRNA internal control. BMY1, beta-amylase 1 (At4g15210); BMY7, beta-amylase 7 (At3g23920); BMY8, beta-amylase 8 (At4g17090); BMY9, beta-amylase 9 (At4g00490); AMY, alpha-amylase (At1g69830); IMY, isoamylase (At2g39930); Phos b, phosphorylase b (At3g29320); P5CS, delta-1-pyrroline 5-carboxylase synthetase (At2g39795); SPS, sucrose-phosphate synthase (AL391222); Hsp70, heat shock protein 70 (At3g12580); Cor78, low-temperature-induced protein 78 (At5g52310); 18S rRNA (At3g41768).

FIG. 2 shows heat shock and cold shock time course experiments. RT-PCR analysis of selected beta-amylase genes was performed under conditions of heat shock and cold shock. Arrowhead shows 18S rRNA internal control.

FIGS. 3A-3B show time course experiments for soluble sugars and starch accumulation under conditions of heat shock and cold shock.

FIGS. 4A-4C show in vitro compatible solute assay for three enzymes. FIG. 4A: SspI. FIG. 4B: G6PDH. FIG. 4C: ADH.

FIG. 5 shows electron transport chain activity in the absence and presence of soluble sugars during heat shock.

FIG. 6 shows electron transport chain activity in the absence and presence of soluble sugars following freezing stress.

FIGS. 7A-7C show time course compatible solute assay (A) SspI, arrowhead shows the 4 and 5 kb bands, (B) G6PDH, (C) ADH.

### DETAILED DISCLOSURE OF THE INVENTION

The subject invention pertains to materials and methods for protecting plants and plant organelles, such as chloroplasts, during thermal (heat and cold) stress, and other forms of environmental stress such as water, salt stress, nutritional stress, aerobic and anaerobic stress, and wounding. It has been discovered that maltose accumulates in the chloroplast in response to acute temperature shock and can act as chloroplast compatible solute for this vital plant cell organelle. In one embodiment of the invention, a plant, plant tissue or plant cell is transformed with a polynucleotide that encodes a protein that produces, or catalyzes the synthesis of, or results in the production of maltose or a maltose alcohol, such as, for example, maltitol. Polynucleotides can be introduced into plant cells using standard techniques known in the art. In one embodiment, the polynucleotide is overexpressed in the plant in order to elevate the level of maltose or maltose alcohol in a plant. In an exemplified embodiment, the polynucleotide encodes a β-amylase enzyme, or an enzymatically active fragment thereof.

In another embodiment of the present invention, a polynucleotide encodes an α-amylase. The polynucleotide can comprise any sequence that encodes a protein that has α-amylase activity or that otherwise results in the production of maltose. Polynucleotide sequences encoding α-amylase can be synthesized as described in U.S. Pat. Nos. 5,460,952; 5,498,832; and 5,712,112.

In another embodiment of the present invention, a polynucleotide encodes a DBE or encodes a protein that has DBE activity or that results in the production of maltose. Polynucleotide sequences encoding DBE are well known in the art (see, for example, U.S. Pat. Nos. 5,912,413 and 6,469,230).

The protein encoded by a polynucleotide of the present invention can be targeted for chloroplast localization. For example, the targeting sequence of a plant β-amylase can be used. The use of a targeting sequence heterologous to the protein encoded by the polynucleotide is also encompassed within the scope of the invention. Numerous examples of other amino acid sequences that when added on or included in a protein result in the transport of the protein to chloroplasts within a cell are known in the art (see, for example; U.S. Pat. No. 6,489,540). For example, a protein can be directed to a chloroplast by incorporating a chloroplast transit peptide sequence, such as those sequences from ADPGPP, 5-enolpyruvyl-3-phosphoshikimic acid synthase (EPSP synthase), and SS RUBISCO, at the N-terminus of the protein when the protein is synthesized. Examples of chloroplast transit sequences are shown in SEQ ID NOs. 27-32.

In those embodiments where a polynucleotide of the present invention encodes a β-amylase, the polynucleotide can comprise any sequence that encodes a protein that has β-amylase activity or that otherwise results in the production of maltose. Polynucleotide sequences encoding β-amylase are well known in the art. U.S. Pat. Nos. 5,762,057, 5,688,684, 5,863,784, and 5,082,781 describe nucleotide sequences encoding a β-amylase enzyme. Also contemplated within the scope of the invention are polynucleotides that encode β-amylase enzymes that have been modified so as to exhibit reduced inhibition by maltose. Also contemplated within the scope of the invention are polynucleotides that encode β-amylase enzymes that exhibit increased thermostability (Yoshigi et al,. 1995; Mikami et al., 1999). In another embodiment, the polynucleotide further comprises a sequence encoding an inducible maltase enzyme which when expressed rapidly converts maltose to glucose.

In one embodiment of the subject method, a plant, planet tissue, or plant cell is transformed with a polynucleotide of the present invention and a transformed plant comprising the polynucleotide is grown from the transformed cell, tissue or plant.

The subject invention also concerns polynucleotide expression constructs comprising a polynucleotide sequence of the present invention. Expression constructs of the invention generally include regulatory elements that are functional in the intended host cell in which the expression construct is to be expressed in. Thus, a person of ordinary skill in the art can select regulatory elements for use in bacterial host cells, yeast host cells, plant host cells, insect host cells, mammalian host cells, and human host cells. Regulatory elements include, for example, promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the invention can comprise a promoter sequence operably linked to the polynucleotide sequence of the invention. Promoters can be incorporated into a polynucleotide using standard techniques known in the art. Multiple copies of promoters or multiple promoters can be used in an expression construct of the invention. In one embodiment, a promoter can be positioned about the same distance from the transcription start site as it is from the transcription start site in its natural genetic environment. Some variation in this distance is permitted without substantial decrease in promoter activity, A transcription start site is typically included in the expression construct.

Overproduction in a plant cell of a protein, such as a β-amylase or an α-amylase, encoded by a polynucleotide of the invention can be accomplished using polynucleotide constructs that have constitutive or inducible promoters operably linked to, for example, polynucleotides encoding β-amylase that are to be transformed into plants. Accordingly, promoters that drive constitutive expression anid promoters that are inducible are contemplated within the scope of the invention. Constitutive promoters that can be used with the present invention include, but are not limited to, cauliflower mosaic virus (CaMV), nopaline synthase, ubiquitin, and actin promoters.

Inducible promoters that can be used with the present invention include those that are regulated by heat shock, freezing, drought, and salinity. Heat shock promoters that can be used with the subject invention include, but are not limited to, those disclosed in U.S. Pat. Nos. 5,447,858 and 6,268,548, as well as the promoter from the Hsp70 gene (Horvath et al., 1993; Gilmour et al., 1998; Lee et al., 1996; and Kim et al., 2002), or the Hsp101 or Hsp17.6 promoter.

Promoters that are inducible by cold temperature (i.e., cor78) are also contemplated for use with the subject invention. For example, the following cold-regulated elements/promoters can be used with the subject invention: (1) cis-acting cold-regulatory element present in all plant cold-regulated promoters as disclosed by Yamaguchi-Shinozaki. et al. (1994); Baker et al. (1994); Jiang et al. (1996); (2) the COR15a promoter (Baker et al., 1994); (3) the COR78/RD29A promoter (Horvath et. al., 1993; Yamaguchi-Shinozaki et al., 994); (4) the COR6.6 promoter (Wang et al, 1995a); (5) the KIN1 promoter (Wang et al., 1995a) genes of Arabidopsis; (6) the BN115 promoter gene of Brassica napus (White et al., 1994); (7) the genes encoding the proteins involved in cold adaptation in Arabidopsis thaliana as disclosed in U.S. Pat. Nos. 5,296,462 and 5,356,816; the COR15b promoter; and the galactinol synthase promoter.

Promoters regulated by drought conditions including, for example, rd29 of Arabidopsis (Yamaguchi-Shinozaki et al., 1993), FL6-55 and FL2-56 (Seki et al., 2001, can also be used with the subject invention. Further, promoters regulated by a combination of stresses are contemplated for used with the subject invention. For example, genes regulated, by freezing drought, and high salinity including, for example, rd29A, cor78, kin1, kin2, cor15a, rd17, and erd10 in transgenic plants (Yamaguchi-Shinozaki, 2001;,Baker et al., 1994; Wang et al., 1995; Iwasaki et al., 1997), can be used with the subject invention.

Organ-specific promoters are also contemplated and include E8 promoter from tomato. Plant viral promoters can also be used, such as for example, the CaMV 35S (including the enhanced CaMV 35S promoter (see, for example U.S. Pat. No. 5,106,739)) or CaMV 19S promoter. Examples of other plant promoters that can be used include, but are not limited to, prolifera promoter, Ap3 promoter, T-DNA 1′- or 2′-promoter of A. tumafaciens, polygalacturonase promoter, chalcone synthase A (CHS-A) promoter from petunia, tobacco PR-1a promoter, alcA gene promoter, pin2 promoter (Xu et al., 1993), maize WipI promoter, maize trpA gene promoter (U.S. Pat. No. 5,625,136), maize CDPK gene promoter, and RUBISCO SSU promoter (U.S. Pat. No. 5,034,322).

Expression constructs of the invention may optionally contain, a transcription termination sequence, a translation termination sequence, a sequence encoding a signal peptide sequence, such as a sequence encoding a chloroplast transit peptide (for targeting a protein to a plant cell chloroplast), and/or enhancer elements. Transcription termination regions can typically be obtained from the 3′ untranslated region of a eukaryotic or viral gene sequence. Transcription termination sequences can be positioned downstream of a coding sequence to provide for efficient termination. Signal peptides are a group of short amino terminal sequences that encode information responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of operably linked signal peptide sequence is contemplated for use with the polypeptides of the invention. Enhancers are cis-acting elements that increase activity of a promoter and can also be included in the expression construct. Enhancer elements are known in the art, and include, but are not limited to, the CaMV 35S enhancer element, maize shrunken-1 enhancer element, cytomegalovirus (CMV) early promoter enhancer element, and the SV40 enhancer element.

DNA sequences which direct polyadenylation of mRNA transcribed from the expression construct can also be included in the expression construct, and include, but are not limited to, an octopine synthase or nopaline synthase signal. The expression constructs of the invention, can also include a polynucleotide sequence that directs transposition of other genes, i.e., a transposon.

Expression constructs of the invention can also include one or more dominant selectable marker genes, including, for example, genes encoding antibiotic resistance and/or herbicide-resistance for selecting transformed cells. Antibiotic-resistance genes can provide for resistance to one or more of the following antibiotics: hygromycin, kanamycin, bleomycin, G418, streptomycin, paromomycin, neomycin, and spectinomycin. Kanamycin resistance can be provided by neomycin phosphotransferase (NPT II). Herbicide-resistance genes can provide for resistance to phosphinothricin acetyltransferase or glyphosate. Other markers used for cell transformation screening include genes encoding β-glucuronidase (GUS), β-galactosidase, luciferase, nopaline synthase, chloramphenicol acetyltransferase (CAT), green fluorescence protein (GFP), or enhanced GFP (Yang et al., 1996).

The subject invention also concerns polynucleotide vectors comprising a polynucleotide sequence of the invention. Unique restriction enzyme sites can be included at the 5′ and 3′ ends of an expression construct or polynucleotide of the invention to allow for insertion into a polynucleotide vector. As used herein, the term “vector” refers to any genetic element, including for example, plasmids, cosmids, chromosomes, phage, virus, and the like, which is capable of replication when associated with proper control elements and which can transfer polynucleotide sequences between cells. Vectors contain a nucleotide sequence that permits the vector to replicate in a selected host cell. A number of vectors are available for expression and/or cloning, and include, but are not limited to, pBR322, pUC series, M13 series, and pBLUESCRIPT vectors (Stratagene, La Jolla, Calif.).

Polynucleotides of the present invention can be composed of either RNA or DNA. Preferably, the polynucleotides are composed of DNA. The subject invention also encompasses those polynucleotides that are complementary in sequence to the polynucleotides disclosed herein.

Because of the degeneracy of the genetic code, a variety of different polynucleotide sequences can encode a protein that produces, or catalyzes the synthesis of, or results in the production of maltose or a maltose alcohol, such as a β-amylase as disclosed herein. In addition, it is well within the skill of a person trained in the art to create alternative polynucleotide sequences encoding the same, or essentially the same, polypeptides of the subject invention. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention.

Substitution of amino acids other than those specifically exemplified or naturally present in a polypeptide encoded by a polynucleotide of the invention are also contemplated within the scope of the present invention. For example, non-natural amino acids can be substituted for the amino acids of the native protein, so long as the protein having the substituted amino acids retains substantially the same biological activity as the native protein in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence are also encompassed within the scope of the invention.

Substitution of amino acids other than those specifically exemplified in a β-amylase or other-proteins of the present invention as disclosed herein are also contemplated within the scope of the present invention. For example, non-natural amino acids can be substituted for the amino acids of the exemplified protein, so long as the protein retains substantially the same biological activity as the exemplified protein. Amino acids can be placed in the following classes: non-polar, uncharged polar, basic, and acidic. Conservative substitutions whereby a protein having an amino acid of one class is replaced with another amino acid of the same class fall within the scope of the subject invention so long as the protein having the substitution still retains substantially the same biological activity as the exemplified protein. Table 1 below provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns variants of the polynucleotides of the present invention that encode a protein that produces, or catalyzes the synthesis of, or results in the production of maltose or a maltose alcohol. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be, prepared and tested using standard, techniques known in the art.

Polynucleotides and proteins of the subject invention can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules having sequences which are sufficiently homologous with the polynucleotide sequences exemplified herein so as to permit hybridization with that sequence under standard stringent conditions and standard methods (Maniatis, T. et al., 1982). As used herein, “stringent” conditions for hybridization refers to conditions wherein hybridization is typically carried out overnight at 20-25° C. below the melting temperature (Tm) of the DNA hybrid in 6×SSPE, 5×Denhardt's solution, 0.1% SDS, 0.1 mg/ml denatured DNA. The melting temperature is described by the following formula (Beltz, G. A. et al., 1983):

Tm=81.5 C+16.6 Log[Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

As used herein, the terms “nucleic acid” and “polynucleotide sequence” refer to a deoxyribonucleotide or ribonucleotide polymer in either single- or double-stranded form, and unless otherwise limited, would encompass known analogs of natural nucleotides that can function in a similar manner as naturally-occurring nucleotides. The polynucleotide sequences include both the DNA strand sequence that is transcribed into RNA and the RNA sequence that is translated into protein. The polynucleotide sequences include both full-length sequences as well as shorter sequences derived from the full-length sequences. It is understood that a particular polynucleotide sequence includes the degenerate codons of the native sequence or sequences which may be introduced to provide codon preference in a specific host cell. Allelic variations of the exemplified sequences also come within the scope of the subject invention. The polynucleotide sequences falling within the scope of the subject, invention further include sequences which specifically hybridize with the exemplified sequences. The polynucleotide includes both the sense and antisense strands as either individual strands or in the duplex.

The subject invention also concerns plants, plant tissue and plant cells transformed with or bred to contain a polynucleotide that encodes a protein that produces, or catalyzes the synthesis of, or results in the production of maltose or a maltose alcohol. In an exemplified embodiment, the polynucleotide encodes a β-amylase enzyme, or an enzymatically active fragment thereof. Optionally, the β-amylase is one that exhibits reduced inhibition by maltose and/or exhibits increased thermostability. Plants, plant tissue and plant cells expressing a polynucleotide of the subject invention exhibit, increased tolerance or resistance to environmental stresses, including heat stress, cold stress, water stress, and salt stress. In one embodiment, the polynucleotide is overexpressed in the plant cell relative to a plant that has not been transformed or bred to contain a polynucleotide of the invention. Preferably, the polynucleotide is expressed primarily during periods of environmental stress, such as heat or cold. Thus, in one embodiment, a plant comprises a polynucleotide of the invention wherein the coding region of the polynucleotide is operably linked to a promoter that is inducible at a particular temperature range, for example, a temperature that would be cold or a temperature that would be hot to a particular species of plant. In another embodiment, a plant comprises a polynucleotide of the invention wherein the coding region of the polynucleotide is operably linked to a cold temperature inducible promoter, and also comprises a polynucleotide of the invention wherein the coding region of the polynucleotide is operably linked to a heat inducible promoter. In a further embodiment, a polynucleotide of the invention comprises a promoter that is both heat and cold inducible.

Plants within the scope of the present invention include monocotyledonous plants, such as rice, wheat, barley, oats, rye, sorghum, maize, lilies, banana, pineapple, turfgrass, gladiolus, and millet, and dicotyledonous plants, such as cotton, peas, alfalfa, chickpea, chicory, clover, kale, lentil, prairie grass, soybean, tobacco, potato, sweet potato, radish, cabbage, rape, apple trees, coffee, tomato, melon, citrus, beans, roses, sugar beet, squash, peppers, strawberry, carnation, chrysanthemums, impatiens, eucalyptus, and lettuce. In one embodiment, the plant is one that is sensitive to or otherwise adversely impacted by exposure to cold and/or hot temperature. Techniques for transforming plants with a gene are known in the art and include, for example, Agrobacterium infection, biolistic methods, etc.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Material and Methods

### Plant Growth and Heat and Cold Shock Treatment

Arabidopsis thaliana growth conditions: Arabidopsis thaliana (var. Colombia) seeds were stratified at 4° C. for 3 days and grown in Fafard 2 mix soil (Canadian Sphagnum Peat (55%), Perlite, and Vermiculite) with a 15/9 hr light/dark cycle at 20° C.+2 for 16-18 days. The light intensity was 25-40 μmol m⁻² s⁻¹ photosynthetically active radiation (PAR). Six days before the experiment, light intensity was increased to 90-140 μmol m⁻² s⁻¹ PAR. Plants were exposed the same light intensity 90-140 μmol m⁻² s⁻¹ PAR during heat and cold shock.

Step-up and a step-down experiments: Sixteen-day old Arabidopsis plants were exposed to 25, 30, 35, 40 and 45° C. for 1 hr for heat shock and 15, 10, 5, and 0° C. for 12 hrs for cold shock and in both experiments, 20° C. grown plants were used as a control. Leaf samples were taken 2 hr after the lights were on for RNA extraction for RT-PCR analyses.

Time course experiment: Eighteen-day old Arabidopsis plants were exposed to 40° C. for 0, 30, 60, 120 and 240 min for heat shock and to 5° C. for 0, 6, 24, 48, 96 and 192 hr for cold shock. Leaf samples were taken 2 hr after the lights were on for RNA extraction for RT-PCR analysis, carbohydrate analysis, western blot, enzyme assay and chlorophyll analysis.

Pea growth conditions: Pea seeds variety Progress #9 (J. W. Jung Seed Company) were soaked in flowing water for about 8 hr before planting. The next day, imbibed seeds were planted on vermiculite and placed in controlled environment at 18° C.±2, 150 μmol m⁻² s⁻¹ PAR with a 12/12 light/dark cycle for 9-10 days.

### RNA Extraction

Heat and cold shocked Arabidospis leaf tissues were flash frozen in liquid N₂ and stored at −80 freezer. Leaf tissues were ground in fine powder in liquid N₂ using pestle and mortar, then extracted using QIAGEN RNeasy Plant Mini Kits (QIAGEN) according to manufacturer's protocol. RLC buffer was used as a lysis buffer in this kit. RNA was quantified using UV spectrophotometry and stored at −80 freezer.

### Reverse Transcriptase-Polymerase Chain Reaction (RT-PCR)

Ready To Go RT-PCR Beads (Amersham Pharmacia Biotech) were used for RT-PCR. Each 25 μl RT-PCR included 1 Unit Taq DNA polymerase, 10 mM pH 9 Tris-HCl, 60 mM KCl, 1.5 mM MgCl₂, 200 μM each dNTP, Moloney Murine Leukemia virus reverse transcriptase, RNAguard RNase inhibitor (porcine), stabilizers, DNase and RNase-free BSA, 1 μg primer de-oxy nucleotide: (pd(N)₆) for the first strand synthesis, 0.4 μM each gene specific forward and reverse primers (Table 2), 0.1 μM each 18S rRNA forward and reverse primer (internal loading control), 0.3 μM each 3′ terminal dideoxy 18S rRNA forward and reverse primers, and 16 ng total RNA. After addition of total RNA and pd(N)₆, each reaction mix was incubated at 42° C. for 30 min for the first strand cDNA synthesis. Then gene specific and 18S rRNA primers (as a loading control) were added and PCR amplification was carried out with a Stratagene Robocycler (Stratagene). PCR cycles were:

first cycle: 95° C. for 5 min, 95° C. for 30 sec, 52° C. for 1 min, 72° C. for 1 min;

second cycle: 95° C. for 30 sec, 52° C. for 1 min, 72° C. for 1 min which is repeated 25-40 times depending on the gene of interest;

final cycle: 72° C. for 7 min.

Then PCR products were kept at 5° C. PCR products were fractionated in a 1% agarose gel. Gels were stained with EtBr and digitally recorded.

TABLE 2
List of primers used in RT-PCR reactions.

| Gene of | | | | |
|---|---|---|---|---|
| Interest | Accession # | Primer # | Primers 5′ to 3′ | |
| BMY1 | At4g15210 | CG363 | ACGCCGGAGAATACAATG (SEQ ID NO. 1) | F |
| | | CG364 | CAACGGCACAATCTCATG (SEQ ID NO. 2) | R |
| BMY7 | At3g23920 | CG305 | GACACCCAGTTCAAAA (SEQ ID NO. 3) | F |
| | | CG306 | CTCAACTTCTTCCCGACA (SEQ ID NO. 4) | R |
| BMY8 | At4g17090 | CG307 | GGAACAAGCGGACCTCAT (SEQ ID NO. 5) | F |
| | | CG308 | TCTCAGCGATCTTGCCTT (SEQ ID NO. 6) | R |
| BMY9 | At4g00490 | CG382 | GCTGGCAGGCGTAACACT (SEQ ID NO. 7) | F |
| | | CG383 | CGGTTTGAGGAGTTGTAGAAG (SEQ ID NO. 8) | R |
| IMY | At2g39930 | CG351 | CGTCTTGAACCACACAGC (SEQ ID NO. 9) | F |
| | | CG352 | GCAAAGTCTCCCTCCTCT (SEQ ID NO. 10) | R |
| AMY | At1g69830 | CG345 | CCAGGGTAGAGGAAACAA (SEQ ID NO. 11) | F |
| | | CG346 | TCGAAGAAGACCGCTGGT (SEQ ID NO. 12) | R |
| PHOS b | At3g29320 | CG315 | AAGATGAAGGAAATGAGTG (SEQ ID NO. 13) | F |
| | | CG316 | CATCTTTTCTGGTCTCGG (SEQ ID NO. 14) | R |
| P5CS | At2g39795 | CG353 | GGACCAAGGGCAAGTAAG (SEQ ID NO. 15) | F |
| | | CG354 | AGCCCATCCTCCTCTGTG (SEQ ID NO. 16) | R |
| SPS | At5g11110 | CG321 | AATGACAATATCTGAGACTC (SEQ ID NO. 17) | F |
| | | CG322 | ACCACATTCTTTAGCCTC (SEQ ID NO. 18) | R |
| RD29A or | At5g52310 | CG309 | CTTTGACTCTGTTCTCGGT (SEQ ID NO. 19) | F |
| Cor78 | | | | |
| | | CG310 | GTTGTCAGTTTCTCCGCC (SEQ ID NO. 20) | R |
| Hsp70 | At3g12580 | CG258 | TCAAGCGGATAAGAGTCACT (SEQ ID NO. 21) | F |
| | | CG259 | CTCGTCCGGGTTAATGCT (SEQ ID NO. 22) | R |
| 18S rRNA | At3g41768 | CG359 | GGAGCGATTTGTCTGGTT (SEQ ID NO. 23) | F |
| | | CG360 | TGATGACTCGCGCTTACT (SEQ ID NO. 24) | R |
| 18S rRNA | At3g41768 | CG361 | GGAGCGATTTGTCTGGTT-3′ (SEQ ID NO. 25) | F |
| 3′ dideoxy | | CG362 | TGATGACTCGCGCTTACT-3′ (SEQ ID NO. 26) | R |

### Chlorophyll Extraction

Chlorophyll content was determined by the method of Bruinsma (1963). Chlorophyll was extracted from 10 mg freeze-dried tissues using 1 ml of 80% acetone at 4° C. overnight in a two ml screw cap micro tube in the dark. Chlorophyll content was quantified spectrophotometrically at 645 and 663 nm.

### Carbohydrate Analysis

Soluble sugar analysis: Eighteen day-old cold and heat stressed Arabidopsis plants were harvested, flash frozen in liquid N₂ and freeze-dried. Lactose 200 μM was added to samples as an internal standard at the beginning of extraction to normalize the data due to losses during the extraction procedure and due to changes in the detection system of HPLC. Soluble sugars were extracted five times using hot 80% aqueous ethanol from 30 mg (dry-weight) of above ground tissue, including leaves and stems. Ethanol insoluble materials were saved for starch analysis. Ethanol was evaporated at 80° C. and the remaining aqueous solution was lyophilized and resuspended in distilled water. To collect soluble neutral sugars, extracts were passed through an Amberlite ion exchange column, which has 1 meq/exchange capacity, at room temperature. The flow-through was freeze-dried. Monosaccharides, which and disaccharides were separated with a NaOH gradient using a Dionex HPLC PA10® column. Monosaccharides, which included fructose and glucose, and disaccharides, which included sucrose, trehalose, maltose and maltitol (sugar alcohol form of maltose), were quantified.

Starch analysis: Starch content was determined by the method of Li et al. (1965). After soluble sugar extraction, the EtOH insoluble residue was vacuum-dried. Starch was solubilized in boiling water for 15 min. The supernatant was reacted with iodine-potassium iodide (0.1%) and color density was measured at 620 nm using spectrophotometer. Potato starch was used as standard to estimate starch quantity.

### Compatible Solute Assay for Maltose

SspI (New England Biolabs) is a bacterial restriction enzyme that cuts double stranded DNA. In this assay it cuts pGEX-4T-2 circular plasmid twice and generates 3.77 kb and 1.19 kb bands in the reaction buffer (50 mM NaCl, 100 mM Tris-HCL, 10I nM MgCl₂, 0.025% Triton X-100 pH 7.5 at 25° C.). Five units of SspI in reaction buffer in the absence of maltose were exposed to 50° C. for 15 min where 50% of the enzyme activity was lost and also exposed to heat temperature in the presence of 14, 100, 200 and 400 mM maltose. Then substrate, 0.5 μg of pGEX-4T-2 circular plasmid (Amersham Biosciences, Piscataway, N.J.), was added to the reaction mixture and incubated at 37 ° C. for 1 hr. The control reaction did not include maltose and was not exposed to heat shock to show the full enzyme activity. To visualize the products, 1% agarose gel was run and 22 μl of total 50 μl reaction was loaded on to gel. The gel was run at 100 V for 30 min and stained with EtBr to visualize the product. The top band was quantified to assess the protection by maltose. Also, the same assay was done using sucrose, trehalose and glucose to compare the ability of maltose as a compatible solute. A time course experiment was conducted where SspI was treated in the absence and in the presence of 400 mM maltose, glucose, sucrose and trehalose at 50° C. for 0, 5, 10, 15, 20 and 25 min.

Glucose-6-phosphate dehydrogenase (G6PDH): Lyophilized G6PDH from Leuconostoc mesenteroides was purchased from Worthington Biochemical Company. G6PDH in 50 mM Tris-HCl pH 7.8 was exposed to 48° C. for 15 min, which reduced activity by 50%. G6PDH in 50 mM Tris-HCl pH 7.8 was heat shocked in the absence and presence of 14, 100, 200, and 400 mM sugars: maltose, trehalose, glucose and sucrose. The control did not include any sugar and was not given a heat shock. The reaction (1.5 ml) included 2.97 mM MgCl₂, 50 mM Tris-HCl pH 7.8, 0.6 mM β-NADP⁺ (freshly prepared), 10 mM Glucose-6-phosphate , and 595 ng heat shocked G6PDH. Production of NADPH was followed for 3 min at 340 nm using Lambda 3A UV/VIS spectrophotometer (PERKIN-ELMER). A time course experiment with two replications was conducted where G6PDH was treated in the absence and in the presence of 400 mM maltose, glucose, sucrose and trehalose at 48° C. for 0, 10, 15, 20 and 25 min.

Alcohol dehydrogenase (ADH): Lyophilized ADH from yeast 300 U/mg was purchased from Calbiochem. ADH in 50 mM potassium phosphate buffer pH 7.6 was exposed to 53.5° C. for 15 min, where loss of 50% activity occurs. ADH in 50 mM potassium phosphate buffer pH 7.6 was heat shocked in the absence and presence of 14, 100, 200, and 400 mM sugars: maltose, trehalose, glucose and sucrose. The control did not include any sugar and was not given a heat shock. Reaction volume 1.5 ml included 333 mM EtOH, 50 mM potassium phosphate pH 7.6, 4.15 mM β-NAD⁺ (freshly prepared), and 500 ng heat shocked ADH. Production of NADH was followed for 20 s at 340 nm using Lambda 3A UV/VIS spectrophotometer (PERKIN-ELMER). A time course experiment was conducted where ADH was treated in the absence and in the presence of 400 mM maltose, glucose, sucrose and trehalose at 53.5° C. for 0, 5, 10, 15, 20 and 25 min.

### Pea Chloroplast Isolation

Pea chloroplast was isolated according to Cline (1993). Aerial portions of 50 g pea plant were chopped into small pieces into 200 ml of ice cold GR-buffer (50 mM HEPES/adjust pH 7.5 with KOH, 0.33 M sorbitol, 1 mM MgCl₂, 1 mM MnCl₂, 2 mM EDTA, 5 mM Na-ascorbate, 1%, BSA) and homogenized using a probe (named PTA 35/2 m). The homogenate was filtered through 1 layer of Mira cloth and centrifuged at 2000×g for 3 min in a swing-out rotor. Pellet was resuspended in GR buffer, overlayed on a Percoll gradient and centrifuged at 2000×g for 15 min in a swing out rotor. Intact plastids were collected, diluted three times with buffer (50 MM HEPES/KOH pH 8, 0.33 M sorbitol), and pelleted 1.500×g for 5 min. The pellet was resuspended in 25 ml of buffer (50 mM HEPES/KOH pH 8, 0.33 M sorbitol) and chlorophyll content was quantified.

### Thylakoid Isolation

Thylakoids were isolated according to Santarius (1996). The isolated chloroplast pellets were ruptured by resuspending at 1 mg/ml in 10 mM Hepes/KOH, pH 8, 5 mM MgCl₂, and incubating 2 min on ice. Then, wash buffer 1:1 (70 mM KCl, 30 mM NaNO₃, 20 mM K₂SO₄, 5 mM MgCl₂, and 5 mM Hepes/KOH pH 7.5) was added and centrifuged 3300×g for 8 min to collect thylakoid membranes. The pellet was washed at 1 mg/ml in wash buffer and centrifuged at 3300×g for 8 min. Thylakoids were resuspended in wash buffer corresponding to 1 mg/ml chlorophyll.

### In Vitro Freezing Stress and Heat Shock of Electron Transport Chain

Heat shock: Aliquots of 0.2 ml thylakoid in wash buffer (70 mM KCl, 30 mM NaNO₃, 20 mM K₂SO₄, 5 mM MgCl₂, and 5 mM Hepes/KOH pH 7.5) corresponding to 0.5 mg/ml chlorophyll were, exposed to heat shock at 40° C. for 4 min in the absence and presence of 2.5, 14, 28, 56, and 112 mM maltose, glucose and trehalose. Full activity of the whole electron transport chain was measured after isolation of thylakoid membranes. Five independent experiments with 3 replications each were done.

Freezing stress: Aliqutots of 0.2 ml thylakoid in wash buffer (70 mM KCl, 30 mM NaNO₃, 20 mM K₂SO₄, 5 mM MgCl₂, and 5 mM Hepes/KOH pH 7.5) corresponding to 0.5 mg/ml chlorophyll were exposed to freezing stress at −15° C. for 20 hrs in absence and presence of 2.5, 14, 28, 56, and 112 mM maltose, glucose and trehalose. Full activity of the whole electron transport chain was measured after isolation of thylakoid membranes. Four independent experiments with three (3) replications each were done.

Whole electron transport chain assay, with DCPIP: This assay was done according to Hipkins and Baker (1986). Assay buffer included 0.1 M sorbitol, 50 mM Hepes-KOH pH 7.6, 5 mM NaCl, 5 mM MgCl₂, 0.1 mM DCPIP. The reaction was mixed after addition of thylakoid corresponding to 25 μg/ml chlorophyll and illuminated for 10 sec at 200 μmol photons m⁻² sec⁻¹ light intensity. Activity of electron transport chain was determined using redox dye DCPIP reduction at 595 nm.

All the experiments were done in three replications, unless otherwise specified.

### EXAMPLE 1

### Heat and Cold Shock Elicit Specific D-amylase Gene Induction

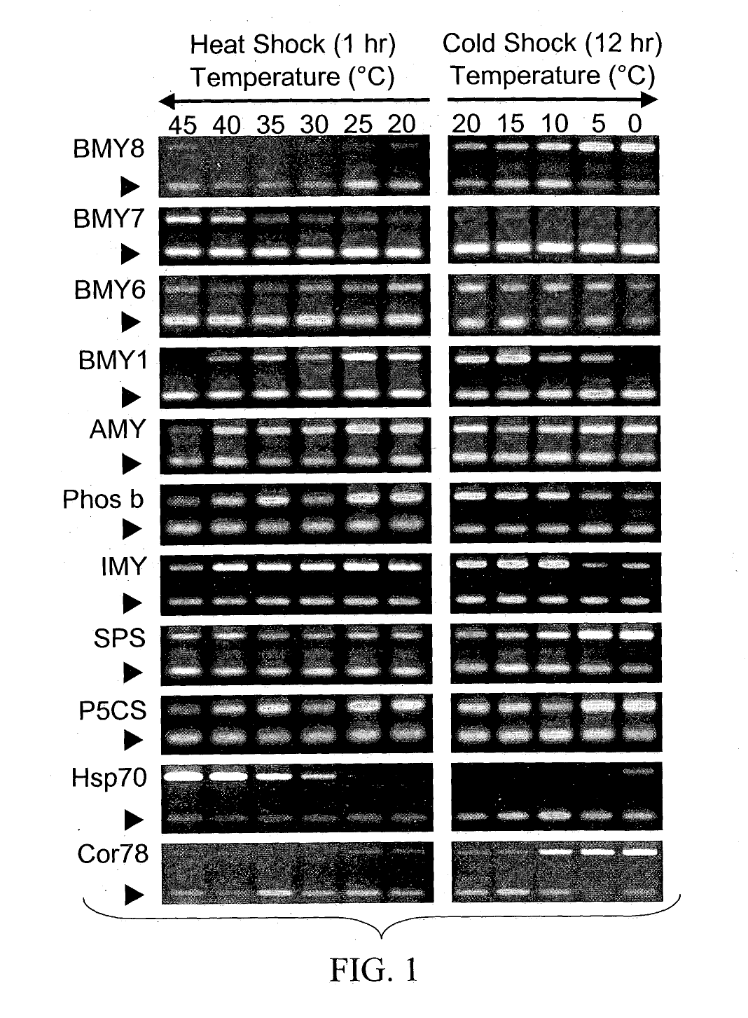

A step-up and step-down experiment was performed to understand how temperature influences β-amylase expression. Plants were exposed to temperatures from 20° to 40° C. for 1 hr in the step-up experiment, and for the step-down experiment, plants were exposed to temperatures from 20° to 0 C for 12 hr (FIG. 1). Gene specific transcript levels were evaluated by RT-PCR. Hsp70 was used Was a control for heat treatment, and its expression gradually increased as temperature increased (FIG. 1). Likewise, Cor78 was used as a control for cold shock treatment and its expression showed slight induction at 10° C. and strong induction at 5 and 0° C. (FIG. 1). BMY8 (FIG. 1) showed the greatest expression at 5° and 0° C. increasing by 15- and 13-fold, respectively. Expression was more modestly induced at 10° C.; approximately 7-fold. BMY8 expression was unchanged under a variety of heat shock temperatures from 20° to 45° C. Conversely, BMY7 expression was markedly increased at 40° and 45° C.; 11- and 8-fold, respectively. Expression was not changed under cold shock temperatures down to 0° C. A third predicted chloroplast-localized beta-amylase (BMY9) did not exhibit any temperature-regulated modulation of expression, but instead was expressed at all temperatures from 45° to 0° C.

The major β-amylase in Arabidopsis is a vacuolar form. For comparison, expression of the vacuolar beta-amylase (BMY1) was also tested and its expression was largely unchanged under heat stress, except at 45° C. where expression became undetectable. BMY1 expression during cold shock decreased 5 fold at 10° and 5° C., and became undetectable at 0° C.

To determine whether modulation of BMY7 and BMY8 expression is a general stress response for starch degrading enzymes, expression profiles fore key enzymes in starch degradation pathways were tested in the step-up, and step-down experiments using RT-PCR (FIG. 1). Alpha-amylase (AMY) was repressed 3-fold at 40° C. and 5-fold at 45° C.; however, its expression was modestly induced 2-fold at 10° and 5° C. Phosphorylase b (Phos b) expression was unchanged under heat stress, repressed 5-fold at 5 and 0° C. Debranching enzyme, isoamylase (IMY) expression was repressed 4-fold at 45° C., but unchanged at all other heat shock temperatures. During cold shock, IMY expression was unchanged except at 0° C. where it was repressed 3-fold. Therefore, induction of beta-amylase BMY7 and BMY8 expression under a variety of heat (from 20° to 45° C.) and cold (from 20° to 0° C.) shock temperatures appears to be a specific response to temperature stress.

Sucrose phosphate synthase (SPS) and delta-1 -pyrroline-5-carboxylate synthetase (P5CS) expression profiles were also compared with beta-amylase expression pattern because their transcripts were known to accumulate during cold shock temperatures. SPS expression levels were gradually increased (FIG. 1), when the temperature was lowered; 3-, 7-, and 10-fold induction at 10°, 5°, and 0° C., respectively. Under heat shock, SPS expression levels were slightly increased. P5CS expression was induced 2.5-fold at 5° and 0° C., but its expression was unchanged under heat shock, except for a slight decrease at 40° and 45° C.(FIG. 1).

### EXAMPLE 2

### Beta-amylase Expression is Correlated with Maltose Accumulation During Time Course

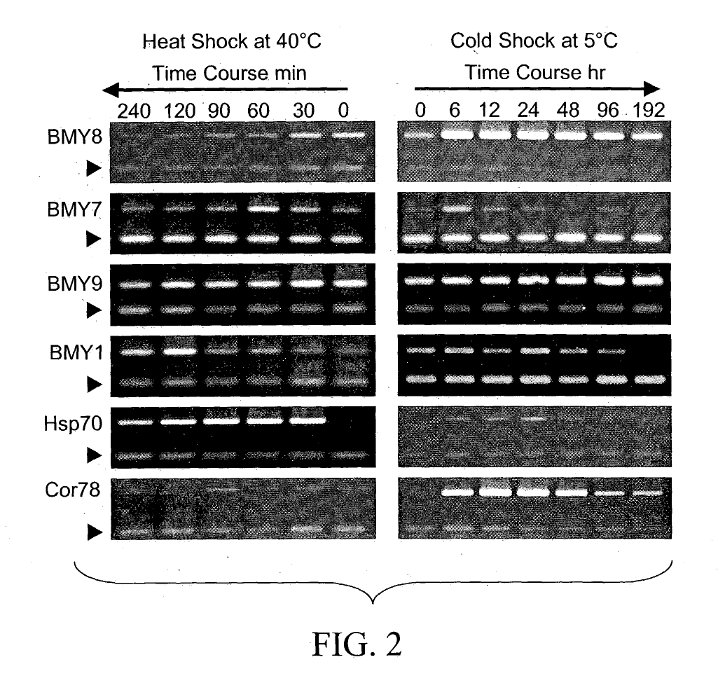

Based on the previous experiment where time was kept constant and plants were exposed to temperature extremes, β-amylase transcripts were found to be most abundant at 40° and 5° C. under heat and cold stress, respectively. RT-PCR (FIG. 2) was performed to show β-amylase transcript levels increase during a time course upon exposure to heat and cold stress. Confirming, the previous. RT-PCR analysis, under cold shock at 5° C., BMY8 expression was induced as early as 6 hr and peaked at 24 hr. then gradually decreased, but still remained higher than control levels at 192 hr. BMY8 expression during heat shock 40° C. was repressed after 60 min. BMY 7 expression peaked at 60 man of exposure to 40° C. and then decreased. It did not show any significant change in expression response to cold shock. FIG. 2, shows that after 6 hr at 5° C., BMY7 expression seemed to be induced, however it was seen only in one replication of the three. BMY9 and BMY1 failed to show, significant changes in expression under heat shock condition for about 2 hours; after that, BMY9 expression slightly decreased and BMY1 expression was induced for 7 fold. At 5° C., BMY9 and BMY1 expression were unaffected. As a control for heat treatment, Hsp70 was used. Its expression peaked at 30-60 min upon exposure to 40° C. Under cold shock conditions, Hsp70 also exhibited a slight induction until 24 hr, after which its expression became undetectable. Hsp70 is known to be induced modestly in response to cold shock. As a cold shock treatment control, Cor 78 was used. It showed a very similar expression profile to BMY8 under cold shock 5° C., but no significant changes under heat shock conditions.

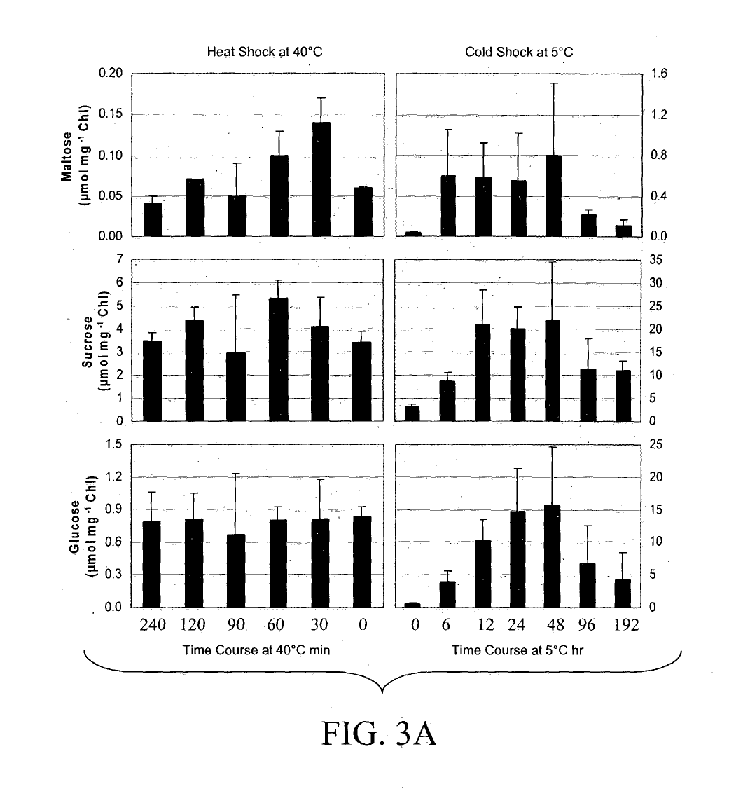

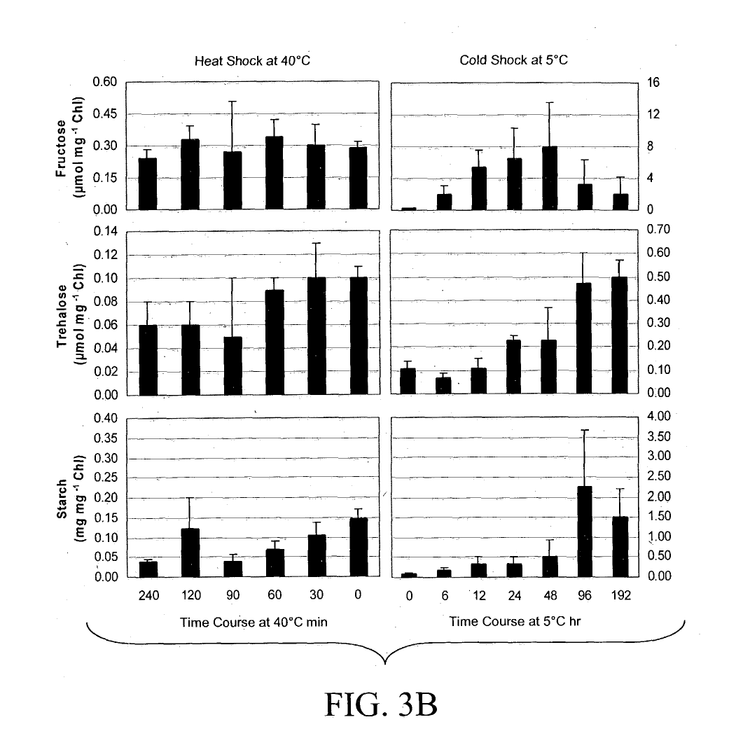

Leaf tissue maltose content was characterized to determine whether maltose accumulation paralleled β-amylase expression. Soluble sugar analysis (FIG. 3) revealed that maltose content doubled from 0.06 to 0.14 βmol mg⁻Ch1 within 30 min exposure to 40° C., remained constant until 60 min, and then gradually decreased back to control levels at 4 hr. The maltose accumulation profile (FIG. 3) was very similar to that of sucrose. During cold shock at 5° C., the maltose content increase was much more dramatic from 0.04 (control) to 0.60 μmol in the first 6 hr then continued to accumulate to 0.80 μmol mg. Ch1 by 48 hr. Afterward it gradually decreased to 0.11 μmol mg⁻¹ Ch1 by 192 hr. The maltose accumulation profile (FIG. 3) was similar to that of sucrose, glucose and fructose. When soluble sugar content began decreasing, trehalose content started increasing about 96 hr after cold shock. In contrast, trehalose content slightly decreased after 90 min of exposure to 40° C.

Intact chloroplast were isolated from 10 day-old pea leaves exposed to 40° C. for 30 min or to 5° C. for 24 hr to show whether maltose actually accumulates in the chloroplast under temperature stress. Maltose content increased in pea chloroplasts by, about 2.5 fold; but overall, the maltose content was about one order of magnitude less than that of Arabidopsis.

Starch content was also determined in order to better understand its relationship with maltose accumulation. Starch content increased gradually and was correlated with accumulation of maltose at about 48 hr during cold shock. Accumulation of starch became pronounced after 96 hr of cold shock; however, this was not followed by maltose accumulation. Conversely, during heat shock at 40° C., starch content decreased over time from 0.15 to 0.04 mg mg⁻¹ Ch1.

### EXAMPLE 3

### Maltose has Compatible Solute Properties

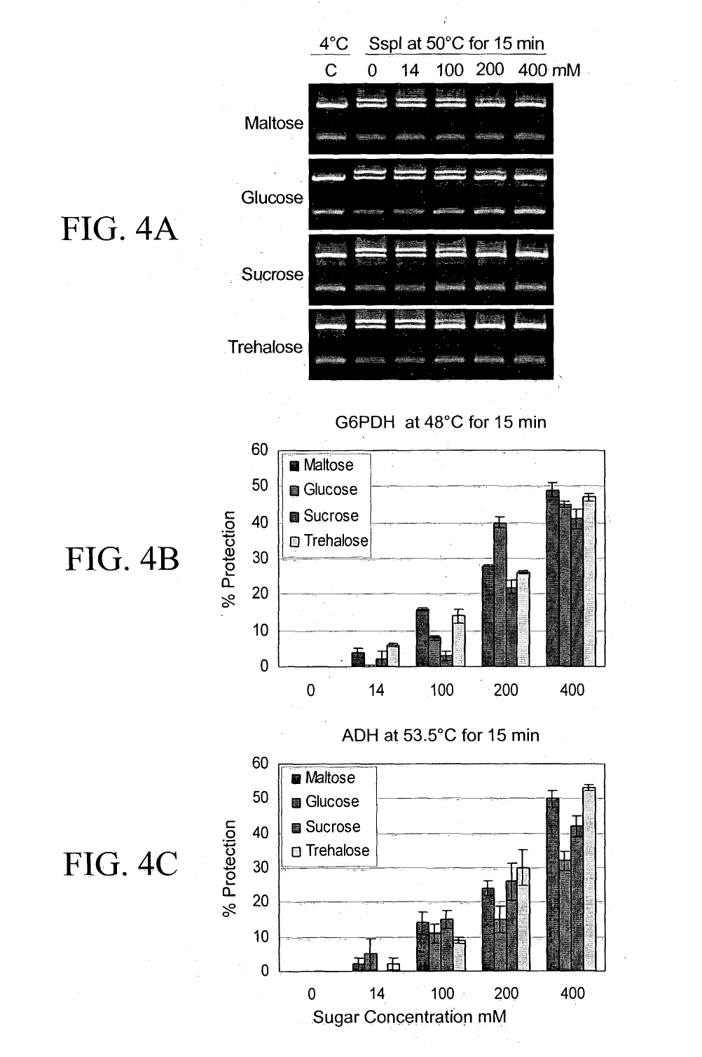

Compatible solutes can stabilize proteins and membranes and contribute to cell osmotic potential during stress. The compatible solute potential of maltose was tested for three different enzymes namely; SspI, Glucose-6-phosphate dehydrogenase (G6PDH) and alcohol dehydrogenase (ADH). The restriction enzyme, SspI (FIG. 4A), cuts the substrate plasmid (pGEX-4T-2; 4.97 kb) twice leading to 1 and 4 kb plasmid fragments, when the enzyme is fully functional. If the enzyme is compromised by high temperature, it does not completely digest the plasmid, but produces a single cut plasmid fragment of 5 kb (top band) band and double cut fragments of 1 and 4 kb (FIG. 4A); as a result, 3 bands are seen instead of 2 bands. When the intensity of the 5 kb top band and the 4 kb band was equal, the enzyme was considered to have lost 50% activity. Sspl was exposed to 50° C. for 15 min (FIG. 4A) in the absence of maltose (0 mM as a control) and in the presence of (14, 100, 200, and 400 mM) maltose. The 5 kb top band, which represented the loss of activity, was quantified to determine relative protection. When the enzyme was fully active, the top band was not visible (FIG. 4A, lane C) and the intensity of the top band increased with increased loss of enzyme activity (lane 2; 0 mM sugars). At a physiological concentration, maltose (14 mM) was able to partially protect SspI function. This protection increased with the increasing maltose concentration (FIG. 4A). At 200 and 400 mM, the intensity of the top band was much less compared to 0 mM maltose.

Besides maltose, identical concentrations of sucrose, trehalose and glucose were also tested to compare performance of maltose as a compatible solute (FIG. 4A). Maltose performed as well as trehalose at physiological concentration. It was the second best after trehalose and superior to glucose and sucrose. At 200 mM, trehalose showed 100% protection, while the other sugars, including maltrose, showed around 80-90% protection. At 400 mM, glucose, trehalose, and sucrose showed 100% protection, while maltose showed around 85-90% protection.

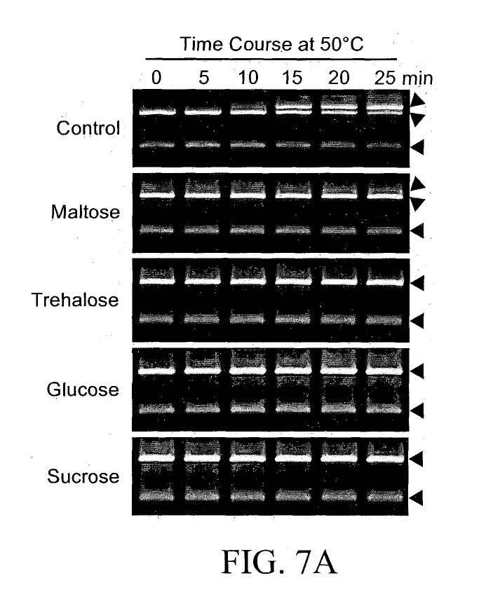

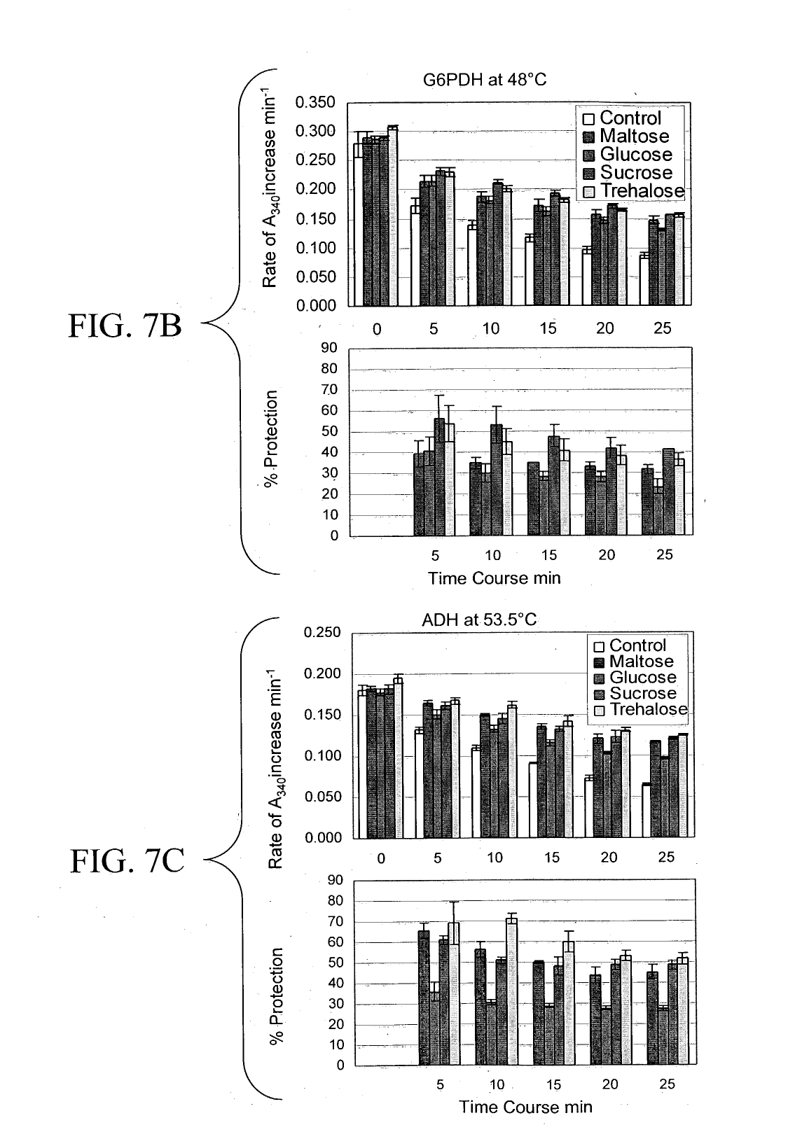

In addition, protection of SspI activity (FIG. 7A) in the presence of the soluble sugars maltose, trehalose, glucose and sucrose (400 mM) can be maintained at least for 25 min at 50° C., while in the absence of sugars the enzyme lost almost 90% activity over time. Trehalose, glucose and sucrose show 100% protection, while maltose exhibited about 90% protection.

To test whether this protection is general, compatible solute assays were performed using G6PDH, which uses D-glucose-6-phosphate and NAD(P)⁺ as substrates to produce D-glucono-δ-lactone-P and NAD(P)H (FIG. 4B). Maltose performed as well as the other compatible solute sugars even at physiological concentration. The same performance was seen (FIG. 7B), when the time was extended to 25 min in the presence of 400 mM of the above sugars.

ADH utilizes RCH₂OH and NAD⁺ as substrates to produce RCHO, NADH and H⁺. ADH was heat stressed (FIG. 4C) in the absence and presence of (14, 100, 200, and 400 mM) maltose, glucose, trehalose, and sucrose. Similar to SspI and G6PDH compatible solute assays, maltose showed a similar level of protection with ADH. The same was true when the time was extended to 25 min at 53.5° C. (FIG. 7C).

### EXAMPLE 4

### Maltose can Function as a Chloroplast Stromal Compatible Solute In Vitro

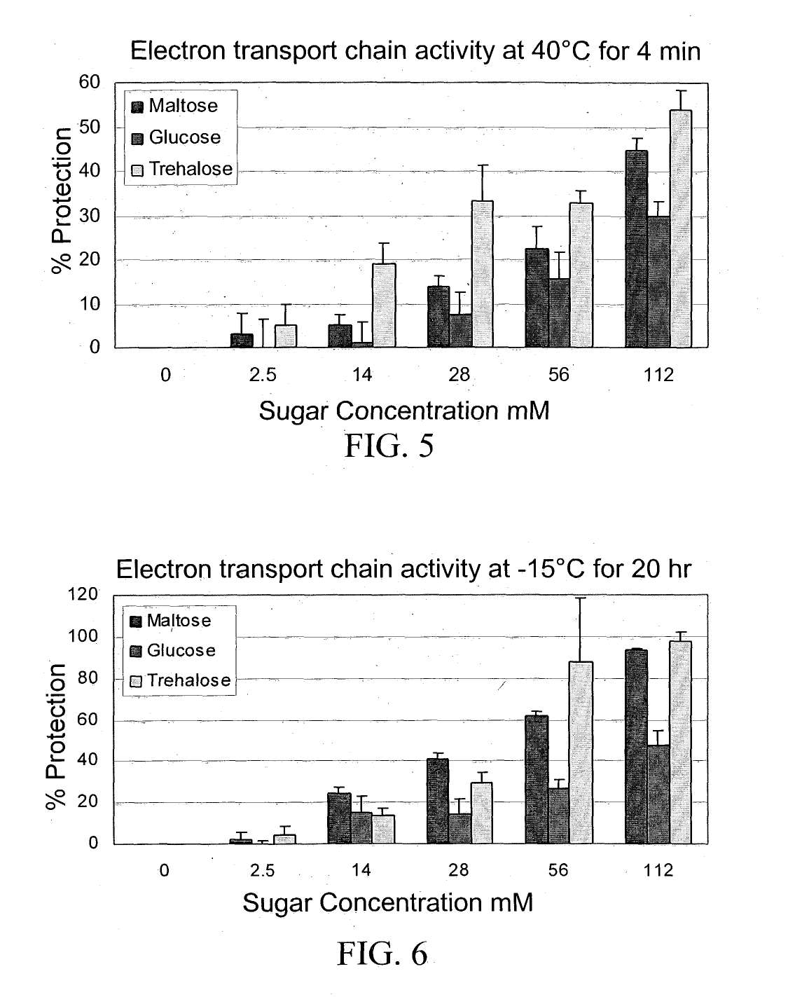

Maltose, trehalose and glucose were tested in vitro for compatible solute properties using thylakoid membranes for the functionality of electron transport chain against heat denaturation (FIG. 5) and freezing stress (FIG. 6). Pea thylakoids were exposed to 40° C. for 4 min (FIG. 5), where 30% of electron transport chain activity remained in the absence of compatible solute sugars. Electron transport chain activity was followed by reduction of a redox dye 2,6-dichlorophenolindophenol (DCPIP), which accepts electrons from Q_{B} of PSII and FeS_{A} of PSI (Häder and Tevini, 1987). At physiological concentrations of 2.5 and 14 mM maltose, photosynthetic electron transport chain activity was protected 3 and 5%. respectively, during heat shock. When the concentration of maltose increased, activity was preserved up to 45% at 112 mM, which is very comparable to the known compatible solute trehalose 54% at the same concentration. Glucose showed 30% protection at 112 mM.

Pea thylakoids were frozen (FIG. 6) at −15° C. for 20 hr, which caused a 60% reduction of electron transport chain activity. Similar to heat shock, protection by maltose was seen at very small maltose concentrations of 2.5 and 14 mM, which preserved 2 and 24% of activity, respectively. This protection increased gradually to 93% with the increasing concentration of maltose and was very similar to that of trehalose. Glucose at 112 mM gave 47% protection, which was much less than that of maltose or trehalose.

It should be understood that the examples and embodiments described herein are for illustrative purposes only and that various modifications or changes in light thereof will be suggested to persons skilled in the art and are to be included within the spirit and purview of this application and the scope of the appended claims.

### References

U.S. Pat. No. 5,762,057

U.S. Pat. No. 5,106,739

U.S. Pat. No. 5,688,684

U.S. Pat. No. 5,863,784

U.S. Pat. No. 5,082,781

U.S. Pat. No. 5,625,136

U.S. Pat. No. 5,034,322

U.S. Pat. No. 5,447 858

U.S. Pat. No. 6,489,540

U.S. Pat. No. 5,460,952

U.S. Pat. No. 5,498,832

U.S. Pat. No. 5,712,112

U.S. Pat. No. 5,912,413

U.S. Pat. No. 6,469,230

U.S. Pat. No. 6,268,548

U.S. Pat. No. 5,296,462

U.S. Pat. No. 5,356,816

Adachi, M., Mikami, B., Katsube, T., Utsumi, S. (1998) “Crystal structure of recombinant soybean β-amylase complexed with β-cyclodextrin” J Biol Chem 273:19859-19865.

Altschul, S. F. et al. (1990) “Basic Local Alignment Search Tool” J Mol. Biol. 215:403-410.

Altschul, S. F. et al. (1997) “Gapped BLAST and PSI-BLAST: A New Generation of Protein Database Search Programs” Nucl. Acids Res. 25:3389-3402.

Avigad, G., Dey, P. M. (1997) “Carbohydrate metabolism: storage carbohydrates” In Plant Biochemistr. P. M. Dey and J. B. Harborne, eds. (Academic Press).

Baker, S. S. et al. (1994) “The 5′-region of Arabidopsis thaliana cor15a has cis-acting elements that confer cold-, drought- and ABA-regulated gene expression” Plant. Mol. Biol. 24:701-713.

Beck, E., Ziegler, P. (1989) “Biosynthesis and degradation of starch in higher plants” Annu. Rev. Plant Physiol, Plant Mol. Biol. 40:95-117.

Beltz, G. A., Jacobs, K. A., Eickbush, T. H., Cherbas, P. T., Kafatos, F. C. (1983) Methods of Enzymology, R. Wu, L. Grossman and K. Moldave [eds.] Academic Press, New York 100:266-285.

Bruinsma, J. (1963) “The quantitative analysis of chlorophylls a and b in plant extracts” Photochem. Photobiol. (Chlor. Metabol. Sym.) 2:241-249.

Cline, K., Henry, R., Li, C. J., Yuan, J. (1993) “Multiple pathways for protein transport into or across the thylakoid membrane” EMBO J. 12:4105-4114.

Datta, R., Selvi, M. T., Seetharama, N., Sharma, R. (1999) “Stress-mediated enhancement of β-amylase activity in pearl millet and maize leaves is dependent on light” J Plant Physiol 1.54:657-664.

Dreier, W., Schnarrenberge, C., Börner, T. (1995) “Light- and stress-dependent enhancement of amylolytic activities in white and green barley leaves: β-amylases are stress-induced proteins” J Plant Physiol 145:342-348.

Gilmour, S. J., Zarka, D. G., .Stockinger, E. J., Salazar, M. P., Houghton, J. M., Thomashow, M. F. (1998) “Low temperature regulation of the Arabidopsis CBF family of AP2 transcriptional activators as an early step in cold-induced COR gene expression” Plant J 16(4):433-442.

Fowler, S., Thomashow, M. F. (2002) “Arabidopsis transcriptome profiling indicates that multiple regulatory pathways are activated during cold acclimation in addition to the CBF cold response pathway” Plant Cell 14:1675-1690.

Gilmour, S., Sebolt, A. M., Salazar, M. P., Everard, J. D., Thomashow, M. F. (2000) “Overexpression of the Arabidopsis CBF3 transcriptional activator mimics multiple biochemical changes associated with cold acclimation” Plant Physiol 124:1854-1865.

Guy, C. L. (1990) “Cold acclimation and freezing tolerance: Role of protein metabolism” Ann. Rev. Plant Physiol. Plant Mol. Biol. 41:187-223.

Guy, C. L., Huber, J. L. A., Huber, S. C. (1992) “Sucrose phosphate synthase and sucrose accumulation at low temperature” Plant Physiol 100:502-508.

Häder, D -P., Tevini, M. (1987) General Photobiology Pergamon Press.

Hipkins and Baker (1986) “Photosynthesis energy transduction a practical approach” Eds Hipkins, M. F. and Baker, N. R. IRL Press, Oxford, England.

Horvath, D. P., MeLamey, B. K. Thomashow, M. F. (1993) “Regulation of Arabidopsis thaliana L. (Heyn) cor78 in response to low temperature” Plant Physiol 103:1047-1053.

Hu, C -A, Delauney, A. J., Verma, D. P. S. (1992) “A bifunctional enzyme (Δ¹-pyrrpline-5-carboxylate synthetase) catalyzes the first two steps in proline biosynthesis in plants” Proc Natl Acad Sci USA 89:9354-9358.

Iwasaki, T. et al. (1997) “The electronic Plant Gene Register” Plant Physiol. 115:1287.

Jiang, C. et al.(1996) “Requirement of a CCGAC cis-acting element for cold induction of the BN115 gene from winter Brassica napus” Plant Mol. Biol. 30:679-684.

Karlin S., Altschul, S. F. (1990) “Methods for Assessing the Statistical Significance of Molecular Sequence Features by Using General Scoring Schemes” Proc. Natl. Acad. Sci. USA 87:2264-2268.

Karlin S., Altschul, S. F. (1993). “Applications and Statistics for Multiple High-Scoring Segments in Molecular Sequences” Proc. Natl. Acad. Sci. USA 90:587.3-5877.

Kim, B. H., Schoffl, F. (2002) “Interaction between Arabidopsis heat shock transcription factor 1 and 70 kDa heat shock proteins” J Exp Bot. 53(367):371-375.

Krapp, A., Stitt, M. (1995) “An evaluation of direct and indirect mechanisms for the “sink-regulation” of photosynthesis in spinach: Changes in, gas exchange, carbohydrates, metabolites, enzyme activities and steady-state transcript levels after cold-girdling source leaves” Planta 195:313-323.

Kreps, J. A., Wu, Y., Chang, S. H., Zhu. T., Wang, X., Harper, J. F. (2002) “Transcriptome changes for Arabidopsis in response to salt; osmotic, and cold stress” Plant Physiol. 130:2129-2141.

Krysan, P. J., Young, J. K., Sussman, M. (1999) “T-DNA as an insertional mutagen in Arabidopsis” Plant Cell 11 2283-2290.

Lao, N. T., Schoneveld, O., Mould, R. M., Hibberd, J. M., Gray, J. C., Kavanagh, T. A. (1999) “An Arabidopsis gene encoding a chloroplast-targeted β-amylase” Plant J 20:519-527.

Lee, J. H., Schoffl, F. (1996) “An Hsp70 antisense gene affects the expression of HSP70/HSC70, the regulation of HSF, and the acquisition of thermotolerance in transgenic Arabidopsis thaliana” Mol Gen Genet. 252(1-2):11-19.

Li, P. H., Weiser, C. J., van Huystee, R. (1965) “Changes in metabolites of red-osier dogwood during cold acclimation” Amer. Soc. Hort. Sci. 86:723-730.

Maniatis, T., Fritsch, E. F., Sambrook, J. (1982) Molecular Cloning: A Laboratory Manual, Cold Spring Harbor Laboratory, Cold Spring Harbor, N.Y.

Mikami, B., Yoon, H. J., Yoshigi, N. (1999) “The crystal structure of the sevenfold mutant of barley beta-amylase with increased thermostability at 2.5 A resolution” J Mol Biol 285:1235-1243.

Monroe, J. D., Preiss, J. (1990) “Purification of a β-amylase that accumulates in Arabidopsis thaliana mutants defective in starch metabolism” Plant Physiol 94:1033-1039.

Nielsen, T. H., Deiting, U., Stitt, M. (1997) “A β-amylase in potato tubers is induced by storage at low temperature” Plant Physiol 113:503-510.

Santarius, K. A. (1973) “The protective effects of sugars on chloroplast membranes during temperature and water stress and its relatioship to frost, desicctaion and heat resistance” Planta 113:105-114.

Santarius, K. A. (1996) “Freezing of isolated thylakoid membranes in complex media. X. Interactions among various low molecular weight cryoprotectants” Cryobiology 33:118-126.

Scheidig, A., Fröhlich A., Schulze, S., Lloyd, J. R., Kossmann, J. (2002) “Downregulation of a chloroplast-targeted β-amylase leads to starch-excess phenotype in leaves” Plant J. 30:581-591.

Seki, M., Narusaka, M., Ishida, J., Nanjo, T., Fujita, M., Oono, Y., Kamiya, A., Nakajima, M., Enju, A., Sakurai, T., Satou, M., Akiyama, K., Taji, T., Yamaguchi-Shinozaki, K., Carninci, P., Kawai, J., Hayashizaki, Y., Shinozaki, K. (2002) “Monitoring the expression profiles of 7000 Arabidopsis genes under drought, cold and high-salinity stresses using a full-length cDNA microarray” Plant J. 31:279-292.

Seki, M., Narusaka, M., Abe, H., Kasuga, M., Yamaguchi-Shinozaki, K., Carninci, P., Hayashizaki, Y., Shinozaki, K. (2001) “Monitoring the expression pattern of 1300 Arabidopsis genes under drought and cold stresses by using a full-length cDNA microarray” Plant Cell. 13:61-72.

Singer, M. A., Lindquist; S. (1998) “Thermotolerance in Saccharomyces cerevisiae: The yin and yang of trehalose” Trends Biotechnol. 16:460-468.

Strand, A., Hurry, . Gustafsson, P., Gardenstrom, P. (1997) “Development of Arabidopsis thaliana leaves at low temperatures releases the suppression of photosynthesis and photosynthetic gene expression despite the accumulation of soluble carbohydrates” Plant J 12:695-614.

Strand, A., Hurry, V., Henkes, S., Huner, N., Gustafsson, P., Gardestrom, P., Stitt, M. (1999) “Acclimation of Arabidopsis leaves developing at low temperatures. increasing cytoplasmic volume accompanies increased activities of enzymes in the Calvin cycle and in the sucrose-biosynthesis pathway” Plant Physiol 119:1387-1397.

Strizhov, N., Abraham, E., Okresz, L., Bickling, S., Zilberstein, A., Schell, J., Koncz, C., Szabados, L., (1997) “Differential expression of two P5CS genes controlling proline accumulation during salt-stress requires ABA and is regulated by ABA1, ABI1, and AXR2 in Arabidopsis” Plant J 12:557-569.

Sung, D -Y., Vierling, E., Guy; C. L. (2001) “Comprehensive expression profile analysis of the Arabidopsis Hsp70 gene gamily” Plant Physiol 126:7.89-800.

Thomashow, M. F. (1999) “Plant cold acclimation: freezing tolerance genes and regulatory mechanisris” Ann. Rev. Plant Physiol. Plant Mol. Biol. 50:571-99.

Todaka, D., Matsushima, H., Morohashi, Y. (2000) “Water stress enhances β-amylase activity in cucumber cotyledons” J Exp Bot 51:739-745.

Verbruggen, N., Villarroel, R., Van Montagu, M. (1993) “Osmoregulation of pyrroline-5-carboxylate reductase gene in Arabidopsis thaliana” Plant Physiol 103:771-781.

Wang, H. et al. (1995) “Promoters from kinI and cor6.6, two homologous Arabidopsis thaliana genes: transcriptional regulation and gene expression induced by low temperature, ABA, osmoticum and dehydration” Plant Mol. Biol. 28:605-617.

Wang, Q., Monroe, J., Sjolund, R. D. (1995) “Identification and characterization of a phloem-specific β-Amylase” Plant Physiol 109:743-750.

Wanner, L. A., Juntilla, O. (1999) “Cold-induced freezing tolerance in Arabidopsis” Plant Physiol 120:391-399.

White, T. C. et al. (1994) “Regulation of BN115, a low-temperature-responsive gene from winter Brassica napus” Plant Physiol. 106:917-928.

Xin, Z., Browse, J. (1998) “eskimol mutants of Arabidopsis are constitutively freezing-tolerant” Proc Natl Acad Sci USA 95:7799-7804.

Xu, D., McElroy, D., Thornburg, R. W., Wu, R. et al. (1993) “Systemic induction of a potato pin2 promoter by wounding, methyl jasmonate, and abscisic acid in transgenic rice plants” Plant Molecular Biology 22:573-588.

Yamaguchi-Shinozaki, et al. (1994) “A novel cis-acting element in an Arabidopsis gene is involved in responsiveness to drought, low-temperature, or high-salt stress” Plant Cell 6:251-264.

Yamaguchi-Shinozaki, K., Shinozaki, K. (1993) “Characterization of the expression of a desiccation-responsive rd29 gene of Arabidopsis thaliana and analysis of its promoter in transgenic plants” Mol Gen Genet 236:331-340.

Yamaguchi-Shinozaki, K. (2001) “Identification of target genes of the DREB1A transcription factor controlling abiotic-stress-responsive gene expression using a full-length cDNA microarray” JIRCAS Annual Report, 46 (Topic 1).

Yancey, P. H., Clark, M. E., Hand, S. C., Bowlus, R. D., Somero, G. N. (1982) “Living with water stress: evolution of osmolyte systems” Science 217:1214-1222.

Yang, T. T. et al. (1996) “Optimized Codon Usage and Chromophore Mutations Provide Enhanced Sensitivity with the Green Fluorescent Protein” Nucleic Acid Research 24(22):4592-4593.

Yoshiba, Y., Kiyosue, T., Katagiri, T., Ueda, H., Mizoguchi, T., Yamaguchi-Shinozaki, K., Wada, K., Harada, Y., Shinozaki, K. (1995) “Correlation between the induction of a gene for Δ¹-pyrroline-5-carboxylate synthetase and the accumulation of proline in Arabidopsis thaliana under osmotic stress” Plant J 7:751-760.

Yoshigi, N., Okada, Y., Maeba, H., Sahara, H., Tamaki, T. (1995) “Construction of a plasmid used for the expression of a sevenfold-mutant, barley beta-amylase with increased thermostability in Escherichia coli and properties of the sevenfold-mutant beta-amylase” J Biochem 118(3):562-567.

We claim:

1. A method for increasing the resistance of a plant to an environmental stress condition, said method comprising introducing a polynucleotide into said plant, wherein said polynucleotide comprises a coding region that encodes a polypeptide that produces, catalyzes the synthesis of, or results in the production of maltose or a maltose alcohol.

2. The method according to claim 1, wherein s ad environmental stress condition is selected from the group consisting of thermal stress, water stress, and salt stress.

3. The method according to claim 1, wherein said thermal stress is heat stress.

4. The method according to claim 1, wherein said thermal stress is cold stress.

5. The method according to claim 1, wherein said polynucleotide encodes an enzyme selected from the group consisting of α-amylase, a β-amylase enzyme, starch phosphorylase, and starch debranching enzyme (DBE), or an enzymatically active fragment thereof.

6. The method according to claim 5, wherein said β-amylase enzyme exhibits reduced inhibition by maltose.

7. The method according to claim 5, wherein said β-amylase enzyme is thermostable.

8. The method according to claim 1, wherein said polypeptide encoded by said polynucleotide comprises an amino acid sequence that targets said polypeptide for chloroplast localization.

9. The method according to claim 1, wherein said polynucleotide comprises a promoter sequence operably linked to said coding region of said polynucleotide.

10. The method according to claim 9, wherein said promoter is an inducible promoter.

11. The method according to claim 10, wherein said inducible promoter is induced by an environmental stress condition selected from the group consisting of heat stress and cold stress.

12. The method according to claim 11, wherein said heat stress inducible promoter is a promoter selected from the group consisting of an Hsp70, Hsp101, and Hsp17.6 promoter.

13. The method according to claim 11, wherein said cold stress inducible promoter is a promoter selected from the group consisting of a Cor78, Cor15b, and galactinol synthase promoter.

14. The method according to claim 9, wherein said promoter drives increased expression of said coding region of said polynucleotide.

15. The method according to claim 1, wherein said plant is a monocot.

16. The method according to claim 15, wherein said monocot is selected from the group consisting of rice, wheat, barley, oats, rye, sorghum, maize, lilies, banana, pineapple, turfgrass, gladiolus, and millet.

17. The method according to claim 1, wherein said plant is a dicot.

18. The method according to claim 17, wherein said dicot is selected from the group consisting of cotton, peas, alfalfa, chickpea, chicory, clover, kale, lentil, prairie grass, soybean, tobacco, potato, sweet potato, radish, cabbage, rape, apple trees, coffee, tomato, melon, citrus, beans, roses, sugar beet, squash, peppers, strawberry, carnation, chrysanthemums, impatiens, eucalyptus, and lettuce.

19. The method according to claim 1, wherein said polypeptide is overexpressed in said plant upon exposure of said plant to said environmental stress condition relative to a plant wherein said polynucleotide has not been introduced.

20. A plant, plant tissue, or plant cell transformed with or bred to contain a polynucleotide that comprises a coding region that encodes a polypeptide that produces, catalyzes the synthesis of, or results in the production of maltose or a maltose alcohol, wherein expression of said polynucleotide in said plant, plant tissue, or plant cell increases the resistance of said plant, plant tissue, or plant cell to an environmental stress condition.

21. The plant, plant tissue, or plant cell according to claim 20, wherein said environmental stress condition is selected from the group consisting of thermal stress, water stress, and salt stress.

22. The plant, plant tissue, or plant cell according to claim 20, wherein said thermal stress is heat stress.

23. The plant, plant tissue, or plant cell according to claim 20, wherein said thermal stress is cold stress.

24. The plant, plant tissue, or plant cell according to claim 20, wherein said polynucleotide encodes an enzyme selected from the group consisting of α-amylase, a β-amylase enzyme, starch phosphorylase, and starch debranching enzyme (DBE), or an enzymatically active fragment thereof.

25. The plant, plant tissue, or plant cell according to claim 24, wherein said β-amylase enzyme exhibits reduced inhibition by maltose.

26. The plant, plant tissue, or plant, cell according to claim 24, wherein said β-amylase enzyme is thermostable.

27. The plant, plant tissue, or plant cell according to claim 20, wherein said polypeptide encoded by said polynucleotide comprises an amino acid sequence that targets said polypeptide for chloroplast localization.

28. The plant, plant tissue, or plant cell according to claim 20, wherein said polynucleotide comprises a promoter sequence operably linked to said coding region of said polynucleotide.

29. The plant, plant tissue, or plant cell according to claim 28, wherein said promoter is an inducible promoter.

30. The plant, plant tissue, or plant cell according to claim 29, wherein said inducible promoter is induced by an environmental stress condition selected from the group consisting of heat stress and cold stress.

31. The plant, plant tissue, or plant cell according to claim 30, wherein said heat stress inducible promoter is a promoter selected from the group consisting of an Hsp70, Hsp101, and Hsp17.6 promoter.

32. The plant, plant tissue, or plant cell according to claim 30, wherein said cold stress inducible promoter is a promoter selected from the group consisting of a Cor78, Cor15b, and galactinol synthase promoter.

33. The plant, plant tissue, or plant cell according to claim 28, wherein said promoter drives increased expression of said coding region of said polynucleotide.

34. The plant, plant tissue, or plant cell according to claim 20, wherein said plant is a monocot.

35. The plant, plant tissue, or plant cell according to claim 34, wherein said monocot is selected from the group consisting of rice, wheat, barley, oats, rye, sorghum, maize, lilies, banana, pineapple, turfgrass, gladiolus, and millet.

36. The plant, plant tissue, or plant cell according to claim 20, wherein said plant is a dicot.

37. The plant, plant tissue, or plant cell according to claim 36, wherein said dicot is selected from the group consisting of cotton, peas, alfalfa,,chickpea, chicory, clover, kale, lentil, prairie grass, soybean, tobacco, potato, sweet potato, radish, cabbage, rape, apple trees, coffee, tomato, melon, citrus, beans, roses, sugar beet, squash, peppers, strawberry, carnation, chrysanthemums, impatiens, eucalyptus, and lettuce.

38. The plant, plant tissue, or plant cell according to claim 20, wherein said polypeptide is overexpressed in said plant upon exposure of said plant to said environmental stress condition relative to a plant wherein said polynucleotide has not been introduced.

39. A plant grown from a plant tissue or plant cell of claim 20.

40. A protein comprising an amino acid sequence having α-amylase, β-amylase, starch phosphorylase, or starch debranching enzyme activity, and operably linked thereto, a heterologous amino acid sequence that targets said protein for chloroplast localization.
